(19) United States
(12) Patent Application Publication
(10) Pub. No.: US 2017/0151175 A1
(43) Pub. Date: Jun. 1, 2017

(54) INFANT NUTRITION WITH LIPID GLOBULES TO INCREASE ENERGY EXPENDITURE AND METABOLIC FLEXIBILITY LATER IN LIFE

(71) Applicant: N.V. Nutricia, Zoetermeer (NL)

(72) Inventors: Annemarie OOSTING, Utrecht (NL); Francina Dorothea KODDE, Utrecht (NL); Marieke ABRAHAMSE-BERKEVELD, Utrecht (NL); Eline Marleen VAN DER BEEK, Utrecht (NL)

(73) Assignee: N.V. Nutricia, Zoetermeer (NL)

(21) Appl. No.: 15/259,881

(22) Filed: Sep. 8, 2016

Related U.S. Application Data
(63) Continuation of application No. 14/966,889, filed on Dec. 11, 2015, now abandoned.
Application No. 14/966,889 is a continuation of application No. 14/435,131, filed on Apr. 10, 2015, now abandoned.

(30) Foreign Application Priority Data
Oct. 12, 2012 (NL) PCT/NL2012/050718

(51) Int. Cl. A61K 9/14; A23L 33/12; A23L 33/115; A61K 31/201; A23L 33/00

(52) U.S. Cl. CPC A61K 9/145; A61K 31/201; A23L 33/30; A23L 33/40; A23L 33/115; A23L 33/12; A23V 2002/00

(57) ABSTRACT

The present invention relates to nutrition for infants and young children with particular lipid globules, resulting in programming the metabolism to an increased energy expenditure and improved mitochondrial functioning later in life when exposed to a high fat, high energy diet.

### CROSS-REFERENCE TO RELATED APPLICATIONS

This application is a Continuation of U.S. application Ser. No. 14/966,889, filed Dec. 11, 2015, which is a Continuation of U.S. application Ser. No. 14/435,131, filed Apr. 10, 2015, which is the National Phase of International Patent Application No. PCT/NL2013/050722, filed Oct. 11, 2013, published on Apr. 17, 2014 as WO 2014/058318 A1, which claims priority to International Patent Application No. PCT/NL2012/050718, filed Oct. 12, 2012. The contents of these applications are herein incorporated by reference in their entirety.

### FIELD OF THE INVENTION

The present invention relates to nutrition for infants and young children, in particular infant formulae, for use in metabolic programming of the body resulting in later in life health effects.

### BACKGROUND OF THE INVENTION

Breast-feeding is the preferred method of feeding infants. However, there are circumstances that make breast-feeding impossible or less desirable. In those cases infant formulae are a good alternative. The composition of modern infant formulae is adapted in such a way that it meets many of the special nutritional requirements of the fast growing and developing infant.

Still improvements can be made towards the constitution of infant milk formulae. Compared to breast fed infants formula fed infants have an increased risk of becoming obese and of obtaining non communicable diseases (NCD) such as cardiovascular diseases and type 2 diabetes, later in life. Early in life feeding has a lasting programming effect on disease risks in adulthood. Obesity and other NCD are major health problems in the Western world and a leading preventable cause of death worldwide, with increasing prevalence in adults and children, and authorities view it as one of the most serious public health problems of the 21st century.

WO 2010/0027258 and WO2010/0027259 relate to infant nutrition with altered fat globule architecture which show a decreased obesity later in life. This effect is thought to occur via an effect on adipocyte development, a process taking place during early infancy. However, the role of energy expenditure, especially thermogenesis, is not adressed.

In adults the use of pharmaceutical or nutritional compositions to increase energy expenditure is known to treat obesity or decrease body weight. Snitker et al, 2009, Am J Clin Nutr 89:45-50 discloses the use of capsinoid treatment in decreasing abdominal adiposity. US 2012/0148588 discloses the use of an antibody or antigen-binding fragment that binds to ActRIIB to increase thermogenic adipocytes. WO 2011/138457 discloses the use of polynucleotides to induce or upregulate expression of UCP1 to treat or prevent a disorder of the energy homeostasis. US 2012/0035274 discloses the use of camphene to increase the expression of UCP genes. US 2012/0039852 discloses the use of a Lactobacillus rhamnosus strain and a prebiotic mixture to increase energy expenditure.

However, such solutions are not suitable for use in young children, especially infants. Decreasing energy expenditure in infants and young children is undesirable, since the energy is needed for ensuring a good growth and development, including, but not limited to, development of adipose tissue. Furthermore, in breast fed infants, energy expenditure is the same or even decreased compared to standard formula fed infants (Butte et al, 1990, Ped. Res. 28: 631-640; Lubetzky et al, 2003, J Pediatr 143: 750-753). Increasing energy expenditure in infants is also for that reason not wanted. Moreover, a correlation between decreased energy expenditure during infancy and later in life obesity cannot be made (Stunkard et al, 1999, Am J Clin Nutr 69:524-530).

### SUMMARY OF THE INVENTION

There is therefore a need to find nutritional components, suitable for infant nutrition, in particular for infant formula, that have no direct increasing effect on energy expediture, but that will result in metabolical programming of the body in such a way that later in life energy expenditure is increased, in particular when exposed to a high fat, high energy Western style diet.

The inventors found the solution in providing nutrition for young children, in particular infants, with a lipid component in the form of large lipid globules and/or lipid globules coated with phospholipids. Standard infant formula comprises small lipid globules with no or a very low amount of phospholipids which is insufficient to cover the lipid globule surface. It was now found, using a guinea pig model, that animals, fed during infancy a diet with a fat component with large lipid globules coated with phospholipids, showed a higher energy expenditure during exposure to a high fat, high energy Western style diet later on. This was observed until a time point corresponding with late adolescence, early adulthood, at a time point that no effects on body weight and fat mass were visible yet, and when compared with control animals fed a standard diet with a fat component with small lipid globules and very low levels of phospholipids. The experimental diet group developed a higher body temperature, i.e. increased thermogenesis, in response to the Western style diet challenge compared to the control group, indicating a higher uncoupling activity in the mitochondria of these animals.

Surprisingly and advantageously, these effects were not observed during exposure of the experimental diets early in life, but only later in life when both test and control animal groups were exposed to the same high fat Western style diet.

Indeed using a mouse model with similar experimental set up, a higher uncoupling protein 3 (UCP3) expression in both the skeletal muscle and the white adipose tissue as well as a higher pyruvate dehydrogenase kinase-isozyme 4 (PDK4) and citrate synthase (CS) activity in white adipose tissue was observed later in life in the group having received at infancy a diet with large fat globules coated with phospholipids after 4 h fasting. UCP3 is indirectly involved in thermogenesis. Furthermore UCP3 has been suggested to play a role in fatty acid metabolism. It was also found that early in life nutrition comprising large lipid globules alone without phospholipid coating resulted in higher UCP3 expression later in life. However the largest effect was observed when the lipid golules were both large in size and comprised a phospholipid coating. In fasted state, PDK4 inhibits the pyruvate dehydrogenase complex which oxidizes pyruvate to Acetyl CoA, and in this way inhibits the glycolysis. The higher PDK4 expression after 4 hours of fasting are indicative of an improved metabolic flexibility, i.e. a faster switch from anabolism to catabolism when switching from fed to fasted state. It was also found that citrate synthase activity in the white adipose tissue was increased in this diet group later in life, implicating also a higher mitochondrial activity as a consequence of early postnatal diet of the present invention. Increased oxidative phosphorilation complex (OXPHOS) activity together with increased mtDNA content further indicate that the mitochondrial density later in life has increased. Together these data showed that early postnatal diet with large lipid globules and/or lipid globules coated with phospholipids advantageously results in a higher mitochondrial activity and/or mitochondrial density when exposed to high fat, high energy Western style diet compared to a standard early postnatal diet. A higher mitochondrial activity and/or density and an increased metabolic flexibility not only protects against adiposity, but also against insulin resistance and diabetes type 2.

Therefore a nutriton for infants or young children, especially an infant formula, comprising a fat component with large lipid globules and/or lipid globules coated with phospholipids beneficially will increase energy expenditure, in particular thermogenesis, later in life, in particular when exposed to a high fat Western style diet.

Therefore an infant nutriton, especially an infant formula, comprising a fat component with large lipid globules and/or lipid globules coated with phospholipids beneficially will program mitochondrial activity and/or density and metabolic flexibility later in life. The infant nutrition of the present invention is in particular beneficial for infants at risk, i.e. exposed to an obesogenic environment, having an overweight or obese or diabetic or gestational diabetic mother, or being born preterm or born term with a low or high birth weight.

### DETAILED DESCRIPTION OF THE INVENTION

The invention thus concerns a method for increasing energy expenditure in a human subject when the human subject has reached an age above 36 months, comprising providing a nutritional composition comprising lipid to the human subject when the human subject has an age of 0 to 36 months, wherein the lipid is present in the nutritional composition in an amount of at least 10 wt % based on dry weight and is in the form of lipid globules, the lipid globules having
a) a volume weighted mode diameter above 1.0 μm and/or
b) a coating of phospholipids, the phospholipids being present in an amount of 0.5 to 20 wt % based on total lipid of the nutritional composition.

In one embodiment the method for increasing energy expenditure is a non-therapeutic or non-medical method.

The invention can also be worded as the use of a composition comprising lipid, or the use of lipid, in the manufacture of a nutritional composition, for increasing energy expenditure in a human subject when the human subject has reached an age above 36 months, by administering the nutritional composition comprising the lipid to the human subject when the human subject has an age of 0 to 36 months, wherein the lipid is present in the nutritional composition in an amount of at least 10 wt % based on dry weight and is in the form of lipid globules, the lipid globules having
a) a volume weighted mode diameter above 1.0 μm and/or
b) a coating of phospholipids, the phospholipids being present in an amount of 0.5 to 20 wt % based on total lipid of the nutritional composition.

The invention can also be worded as a nutritional composition comprising lipid, wherein the lipid is present in the nutritional composition in an amount of at least 10 wt % based on dry weight and is in the form of lipid globules, the lipid globules having
a) a volume weighted mode diameter above 1.0 μm and/or
b) a coating of phospholipids, the phospholipids being present in an amount of 0.5 to 20 wt % based on total lipid of the nutritional composition
for use in increasing energy expenditure in a human subject when the human subject has reached an age above 36 months, by administering the nutritional composition comprising lipid to the human subject when the human subject has an age of 0 to 36 months.

In a further embodiment the invention concerns a method for increasing expression and/or activity of
(1) uncoupling protein (UCP),
(2) pyruvate dehydrogenase kinase-isozyme 4 (PDK4)
(3) citrate synthase (CS) and/or
(4) oxidative phosphorylation complex (OXPHOS), preferably
(1) uncoupling protein (UCP),
(2) pyruvate dehydrogenase kinase-isozyme 4 (PDK4) and/or
(3) citrate synthase (CS) in a human subject when the human subject has reached an age above 36 months comprising providing a nutritional composition comprising lipid to the human subject when the human subject has an age of 0 to 36 months, wherein the lipid is present in the nutritional composition in an amount of at least 10 wt % based on dry weight and is in the form of lipid globules, the lipid globules having
a) a volume weighted mode diameter above 1.0 μm and/or
b) a coating of phospholipids, the phospholipids being present in an amount of 0.5 to 20 wt % based on total lipid of the nutritional composition.

In one embodiment the method for increasing expression and/or activity of (1) uncoupling protein, (2) pyruvate dehydrogenase kinase-isozyme 4 (PDK4), (3) citrate synthase (CS) and/or (4) oxidative phosphorylation complex (OXPHOS) is a non-therapeutic or non-medical method.

The invention can also be worded as the use of a composition comprising lipid, or the use of lipid, in the manufacture of a nutritional composition, for increasing expression and/or activity of
(1) uncoupling protein (UCP),
(2) pyruvate dehydrogenase kinase-isozyme 4 (PDK4)
(3) citrate synthase (CS) and/or
(4) oxidative phosphorylation complex (OXPHOS), preferably
(1) uncoupling protein (UCP),
(2) pyruvate dehydrogenase kinase-isozyme 4 (PDK4) and/or
(3) citrate synthase (CS)
in a human subject when the human subject has reached an age above 36 months, by administering the nutritional composition comprising the lipid to the human subject when the human subject has an age of 0 to 36 months, wherein the lipid is present in the nutritional composition in an amount of at least 10 wt % based on dry weight and is in the form of lipid globules, the lipid globules having
a) a volume weighted mode diameter above 1.0 μm and/or
b) a coating of phospholipids, the phospholipids being present in an amount of 0.5 to 20 wt % based on total lipid of the nutritional composition.

The invention can also be worded as a nutritional composition comprising lipid, wherein the lipid is present in the nutritional composition in an amount of at least 10 wt % based on dry weight and is in the form of lipid globules, the lipid globules having
a) a volume weighted mode diameter above 1.0 μm and/or
b) a coating of phospholipids, the phospholipids being present in an amount of 0.5 to 20 wt % based on total lipid of the nutritional composition for use in increasing expression and/or activity of
(1) uncoupling protein (UCP),
(2) pyruvate dehydrogenase kinase-isozyme 4 (PDK4)
(3) citrate synthase (CS) and/or
4) oxidative phosphorylation complex (OXPHOS), preferably
(1) uncoupling protein (UCP),
(2) pyruvate dehydrogenase kinase-isozyme 4 (PDK4) and/or
(3) citrate synthase (CS) in a human subject when the human subject has reached an age above
36 months, by administering the nutritional composition comprising lipid to the human subject when the human subject has an age of 0 to 36 months.

In one embodiment the method for increasing expression and/or activity of (1) uncoupling protein, (2) pyruvate dehydrogenase kinase-isozyme 4 (PDK4), (3) citrate synthase (CS) and/or (4) oxidative phosphorylation complex (OXPHOS) in the human subject is for increasing energy expenditure in the human subject. In a further embodiment the increased energy expenditure is selected from the group consisting of an increased resting energy expenditure, increased thermogenesis, and increased non-exercise associated thermogenesis.

Preferably the uncoupling protein is selected from the group consisting of uncoupling protein 1 (UCP 1) and uncoupling protein 3 (UCP).

In a further embodiment, the invention concerns a method for increasing mitochondrial density and/or increasing metabolic flexibility, preferably for increasing metabolic flexibility, in a human subject when the human subject has reached an age above 36 months comprising providing a nutritional composition comprising lipid to the human subject when the human subject has an age of 0 to 36 months, wherein the lipid is present in the nutritional composition in an amount of at least 10 wt % based on dry weight and is in the form of lipid globules, the lipid globules having
a) a volume weighted mode diameter above 1.0 μm and/or
b) a coating of phospholipids, the phospholipids being present in an amount of 0.5 to 20 wt % based on total lipid of the nutritional composition.

In one embodiment the method for increasing mitochondrial density and/or increasing metabolic flexibility is a non-therapeutic or non-medical method.

This embodiment can also be worded as the use of a composition comprising lipid, or the use of lipid, in the manufacture of a nutritional composition, for increasing mitochondrial density and/or increasing metabolic flexibility, preferably for increasing metabolic flexibility, in a human subject when the human subject has reached an age above 36 months, by administering the nutritional composition comprising the lipid to the human subject when the human subject has an age of 0 to 36 months, wherein the lipid is present in the nutritional composition in an amount of at least 10 wt % based on dry weight and is in the form of lipid globules, the lipid globules having
a) a volume weighted mode diameter above 1.0 μm and/or
b) a coating of phospholipids, the phospholipids being present in an amount of 0.5 to 20 wt % based on total lipid of the nutritional composition.

The invention can also be worded as a nutritional composition comprising lipid, wherein the lipid is present in the nutritional composition in an amount of at least 10 wt % based on dry weight and is in the form of lipid globules, the lipid globules having
a) a volume weighted mode diameter above 1.0 μm and/or
b) a coating of phospholipids, the phospholipids being present in an amount of 0.5 to 20 wt % based on total lipid of the nutritional composition
for use in increasing mitochondrial density and/or increasing metabolic flexibility, preferably for increasing metabolic flexibility, in a human subject when the human subject has reached an age above 36 months, by administering the nutritional composition comprising lipid to the human subject when the human subject has an age of 0 to 36 months.

In one embodiment, the method for increasing mitochondrial density and/or increasing metabolic flexibility in the human subject is for increasing energy expenditure in the human subject. In a further embodiment the increased energy expenditure is selected from the group consisting of an increased resting energy expenditure, increased thermogenesis, and increased non-exercise associated thermogenesis.

In one embodiment “increasing” as in increasing energy expenditure, increasing expression and/or activity of UCP, PDK4 CS and/or OXPHOS, and increasing mitochondrial density and/or increasing metabolic flexibility is with respect to the values obtained for a nutritional composition not comprising lipid globules having a volume weighted mode diameter above 1.0 in particular with respect to a nutritional composition comprising lipid globules having a volume weighted mode diameter below 1.0

In one embodiment “increasing” as in increasing energy expenditure, increasing expression and/or activity of UCP, PDK4, CS and/or OXPHOS, and increasing mitochondrial density and/or increasing metabolic flexibility is with respect to the values obtained for a nutritional composition not comprising phospholipid coated lipid globules.

For sake of clarity it is noted that the present invention is defined in terms of specific ingredients, hence the lipids and phospholipids and by the way these ingredients are assembled, hence as phospholipid coated lipid globules of a certain size. Hence the ingredients and the way they are assembled overlap.

Throughout the description wherever the phrase ‘the present composition’ is used it is to be understood that this refers to the composition that is used in the method according to the present invention or in other words for the use to achieve the specified effect(s).

Lipid Component

The present composition comprises lipid. The lipid provides preferably 30 to 60% of the total calories of the composition. More preferably the present composition comprises lipid providing 35 to 55% of the total calories, even more preferably the present composition comprises lipid providing 40 to 50% of the total calories. When in liquid form, e.g. as a ready-to-feed liquid, the composition preferably comprises 2.1 to 6.5 g lipid per 100 ml, more preferably 3.0 to 4.0 g per 100 ml. Based on dry weight the present composition preferably comprises 10 to 50 wt. %, more preferably 12.5 to 40 wt. % lipid, even more preferably 19 to 30 wt. % lipid.

Lipids include polar lipids (such as phospholipids, glycolipids, sphingomyelin, and cholesterol), monoglycerides, diglycerides, triglycerides and free fatty acids. Preferably the composition comprises at least 75 wt. %, more preferably at least 85 wt. % triglycerides based on total lipids.

The lipid of the present invention preferably comprises vegetable lipids. The presence of vegetable lipids advantageously enables an optimal fatty acid profile, high in (poly)unsaturated fatty acids and/or more reminiscent to human milk fat. Using lipids from cow's milk alone, or other domestic mammals, does not provide an optimal fatty acid profile. Preferably the present composition comprises at least one, preferably at least two lipid sources selected from the group consisting of linseed oil (flaxseed oil), rape seed oil (such as colza oil, low erucic acid rape seed oil and canola oil), salvia oil, perilla oil, purslane oil, lingonberry oil, sea buckthorn oil, hemp oil, sunflower oil, high oleic sunflower oil, safflower oil, high oleic safflower oil, olive oil, black currant seed oil, echium oil, coconut oil, palm oil and palm kernel oil. Preferably the present composition comprises at least one, preferably at least two lipid sources selected from the group consisting of linseed oil, canola oil, coconut oil, sunflower oil and high oleic sunflower oil. Commercially available vegetable lipids are typically offered in the form a continuous oil phase. When in liquid form, e.g. as a ready-to-feed liquid, the composition preferably comprises 2.1 to 6.5 g vegetable lipid per 100 ml, more preferably 3.0 to 4.0 g per 100 ml. Based on dry weight the present composition preferably comprises 10 to 50 wt. %, more preferably 12.5 to 40 wt. % vegetable lipid, even more preferably 19 to 30 wt. %. Preferably the composition comprises 50 to 100 wt. % vegetable lipids based on total lipids, more preferably 70 to 100 wt. %, even more preferably 75 to 97 wt. %. It is noted therefore that the present composition also may comprise non-vegetable lipids. Suitable and preferred non-vegetable lipids are further specified below.

Polar Lipids

The present invention preferably comprises polar lipids. Polar lipids are amphipathic of nature and include glycerophospholipids, glycosphingolipids, sphingomyelin and/or cholesterol. More preferably the composition comprises phospholipids (the sum of glycerophospholipids and sphingomyelin). Polar lipids in the present invention relate to the sum of glycerophospholipids, glycosphingolipids, sphingomyelin and cholesterol. In an embodiment according to the present invention, polar lipids are present as a coating of the lipid globule. By ‘coating’ is meant that the outer surface layer of the lipid globule comprises polar lipids, whereas these polar lipids are virtually absent in the core of the lipid globule. The presence of polar lipids as a coating or outer layer of the lipid globule in the diet administered early in life was found to advantageously result in an increased energy expenditure later in life when exposed to a Western style diet.

The present composition preferably comprises glycerophospholipids. Glycerophospholipids are a class of lipids formed from fatty acids esterified at the hydroxyl groups on carbon-1 and carbon-2 of the backbone glycerol moiety and a negatively-charged phosphate group attached to carbon-3 of the glycerol via an ester bond, and optionally a choline group (in case of phosphatidylcholine, PC), a serine group (in case of phosphatidylserine, PS), an ethanolamine group (in case of phosphatidylethanolamine, PE), an inositol group (in case of phosphatidylinositol, PI) or a glycerol group (in case of phosphatidylglycerol, PG) attached to the phosphate group. Lysophospholipids are a class of phospholipids with one fatty acyl chain. Preferably the present composition contains PC, PS, PI and/or PE, more preferably at least PC.

The present composition preferably comprises phosphospingolipids, preferably sphingomyelin. Sphingomyelins have a phosphorylcholine or phosphorylethanolamine molecule esterified to the 1-hydroxy group of a ceramide. They are classified as phospholipid as well as sphingolipid, but are not classified as a glycerophospholipid nor as a glycosphingolipid. In one embodiment according to the present invention, the nutritional composition comprises at least 0.1 wt % sphingomyelin based on total lipid of the nutritional composition.

The present composition preferably comprises glycosphingolipids. The term glycosphingolipids as in the present invention particularly refers to glycolipids with an amino alcohol sphingosine. The sphingosine backbone is 0-linked to a charged headgroup such as ethanolamine, serine or choline backbone. The backbone is also amide linked to a fatty acyl group. Glycosphingolipids are ceramides with one or more sugar residues joined in a β-glycosidic linkage at the 1-hydroxyl position. Preferably the present composition contains gangliosides, more preferably at least one ganglioside selected from the group consisting of GM3 and GD3.

Sphingolipids are in the present invention defined as the sum of sphingomyelin and glycosphingolipids. Phospholipids are in the present invention defined as the sum of sphingomyelin and glycerophospholipids. Preferably the phospholipids are derived from milk lipids. Preferably the weight ratio of phospholipids:glycosphingolipids is from 2:1 to 10:1, more preferably 2:1 to 5:1.

Preferably the present composition comprises phospholipids. Preferably the present composition comprises 0.5 to 20 wt. % phospholipids based on total lipid, more preferably 0.5 to 10 wt. %, more preferably 1 to 10 wt. %, even more preferably 2 to 10 wt. % even more preferably 3 to 8 wt. % phospholipids based on total lipid. Preferably the present composition comprises 0.1 to 10 wt. % glycosphingolipids based on total lipid, more preferably 0.5 to 5 wt. %, even more preferably 2 to 4 wt %. Preferably the present composition comprises 0.5 to 10 wt. % (glycosphingolipids plus phospholipids) based on total lipid, more preferably 1.0 to 10 wt. % (glycosphingolipids plus phospholipids) based on total lipid.

The present composition preferably comprises cholesterol. The present composition preferably comprises at least 0.005 wt. % cholesterol based on total lipid, more preferably at least 0.02 wt. %, more preferably at least 0.05 wt. %., even more preferably at least 0.1 wt. %. Preferably the amount of cholesterol does not exceed 10 wt. % based on total lipid, more preferably does not exceed 5 wt. %, even more preferably does not exceed 1 wt. % of total lipid.

Preferably the present composition comprises 0.6 to 25 wt. % polar lipids based on total lipid, wherein the polar lipids are the sum of phospholipids, glycosphingolipids, and cholesterol, more preferably 0.6 to 12 wt. %, more preferably 1 to 10 wt. %, even more preferably 2 to 10 wt %, even more preferably 3 to 10 wt. % polar lipids based on total lipid, wherein the polar lipids are the sum of phospholipids, glycosphingolipids, and cholesterol.

Preferred sources for providing the phospholipids, glycosphingolipids and/or cholesterol are egg lipids, milk fat, buttermilk fat and butter serum fat (such as beta serum fat). A preferred source for phospholipids, particularly PC, is soy lecithin and/or sunflower lecithin. The present composition preferably comprises phospholipids derived from milk. Preferably the present composition comprises phospholipids and glycosphingolipids derived from milk. Preferably also cholesterol is obtained from milk. Preferably the polar lipids are derived from milk. Polar lipids derived from milk include the polar lipids isolated from milk lipid, cream lipid, butter serum lipid (beta serum lipid), whey lipid, cheese lipid and/or buttermilk lipid. Buttermilk lipid is typically obtained during the manufacture of buttermilk. Butter serum lipid or beta serum lipid is typically obtained during the manufacture of anhydrous milk fat from butter. Preferably the phospholipids, glycosphingolipids and/or cholesterol are obtained from milk cream. The composition preferably comprises phospholipids, glycosphingolipids and/or cholesterol from milk of cows, mares, sheep, goats, buffalos, horses or camels. It is most preferred to use a lipid extract isolated from cow's milk. The use of polar lipids from milk fat advantageously comprises the polar lipids from milk fat globule membranes, which are more reminiscent to the situation in human milk. Polar lipids derived from fat milk advantageously effect energy expenditure later in life to a larger extent than polar lipids from other sources. The polar lipids are located on the surface of the lipid globule, i.e. as a coating or outer layer. A suitable way to determine whether the polar lipids are located on the surface of the lipid globules is laser scanning microscopy. The concomitant use of polar lipids derived from domestic animals milk and trigycerides derived from vegetable lipids therefore enables to manufacture coated lipid globules with a coating more similar to human milk, while at the same time providing an optimal fatty acid profile. Suitable commercially available sources for milk polar lipids are BAEF, SM2, SM3 and SM4 powder of Corman, Salibra of Glanbia, and LacProdan MFGM-10 or PL20 from Arla. Preferably the source of milk polar lipids comprises at least 4 wt. % phospholipids based on total lipid, more preferably 7 to 75 wt. %, most preferably 20 to 70 wt. % phospholipids based on total lipid. Preferably the weight ratio phospholipids to protein is above 0.10, more preferably above 0.20, even more preferably above 0.3. Preferably at least 25 wt. %, more preferably at least 40 wt. %, most preferably at least 75 wt. % of the polar lipids is derived from milk polar lipids.

Fatty Acid Composition

Herein LA refers to linoleic acid and/or acyl chain (18:2 n6); ALA refers to a-linolenic acid and/or acyl chain (18:3 n3); LC-PUFA refers to long chain polyunsaturated fatty acids and/or acyl chains comprising at least 20 carbon atoms in the fatty acyl chain and with 2 or more unsaturated bonds; DHA refers to docosahexaenoic acid and/or acyl chain (22:6, n3); EPA refers to eicosapentaenoic acid and/or acyl chain (20:5 n3); ARA refers to arachidonic acid and/or acyl chain (20:4 n6); DPA refers to docosapentaenoic acid and/or acyl chain (22:5 n3). Medium chain fatty acids (MCFA) refer to fatty acids and/or acyl chains with a chain length of 6, 8 or 10 carbon atoms.

LA preferably is present in the nutritional composition in a sufficient amount in order to promote a healthy growth and development, yet in an amount as low as possible in view of an unwanted high n6/n3 ratio. The composition therefore preferably comprises less than 15 wt. % LA based on total fatty acids, preferably between 5 and 14.5 wt. %, more preferably between 6 and 10 wt. %. Preferably the composition comprises over 5 wt. % LA based on fatty acids. Preferably ALA is present in the nutritional composition in a sufficient amount to promote a healthy growth and development of the infant. The present composition therefore preferably comprises at least 1.0 wt. % ALA based on total fatty acids. Preferably the composition comprises at least 1.5 wt. % ALA based on total fatty acids, more preferably at least 2.0 wt. %. Preferably the composition comprises less than 10 wt. % ALA, more preferably less than 5.0 wt. % based on total fatty acids. The weight ratio LA/ALA should be well balanced ensuring a normal growth and development. Therefore, the present composition preferably comprises LA and ALA in a weight ratio of LA/ALA between 1 and 15, preferably between 2 and 15, more preferably between 1 and 10, more preferably between 2 and 7, more preferably between 3 and 7, more preferably between 4 and 7, more preferably between 3 and 6, even more preferably between 4 and 5.5, even more preferably between 4 and 5.

The present composition preferably comprises at least 3 wt. % MCFA based on total fatty acids, more preferably at least 10 wt. %, even more preferably 15 wt. %. The present composition advantageously comprises less than 50 wt. % MCFA based on total fatty acids, more preferably less than 40 wt. %, even more preferably less than 25 wt. %.

Preferably the present composition comprises n3 LC-PUFA. More preferably, the present composition comprises EPA, DPA and/or DHA, even more preferably DHA. Since a low concentration of DHA, DPA and/or EPA is already effective and normal growth and development are important, the content of n3 LC-PUFA in the present composition, preferably does not exceed 15 wt. % of the total fatty acid content, preferably does not exceed 10 wt. %, even more preferably does not exceed 5 wt. %. Preferably the present composition comprises at least 0.2 wt. %, preferably at least 0.5 wt. %, more preferably at least 0.75 wt. %, n3 LC-PUFA of the total fatty acid content. In one embodiment the present composition preferably comprises DHA in an amount of 0.1 to 0.6 wt. % based on total fatty acid content.

As the group of n6 fatty acids, especially arachidonic acid (ARA) and LA as its precursor, counteracts the group of n3 fatty acids, especially DHA and EPA, and ALA as their precursor, the present composition comprises relatively low amounts of ARA. The n6 LC-PUFA content preferably does not exceed 5 wt. %, more preferably does not exceed 2.0 wt. %, more preferably does not exceed 0.75 wt. %, even more preferably does not exceed 0.5 wt. %, based on total fatty acids. The amount of n6 LC-PUFA is preferably at least 0.02 wt. % more preferably at least 0.05 wt. %, more preferably at least 0.1 wt. % based on total fatty acids, more preferably at least 0.2 wt. %. The presence of ARA is advantageous in a composition low in LA since it remedies LA deficiency. The presence, preferably of low amounts, of ARA is beneficial in nutrition to be administered to infants below the age of 6 months, since for these infants the infant formulae is generally the only source of nutrition. In one embodiment the present composition preferably comprises ARA in an amount of 0.1 to 0.6 wt. % based on total fatty acid content.

Preferably in addition to the vegetable lipid, a lipid selected from fish oil (preferably tuna fish oil) and single cell oil (such as algal, microbial oil and fungal oil) is present. These sources of oil are suitable as LC-PUFA sources. Preferably as a source of n3 LC-PUFA single cell oil, including algal oil and microbial oil, is used, since these oil sources have an advantageous EPA/DHA ratio. More preferably fish oil (even more preferably tuna fish oil) is used as a source of n3 LC-PUFA since fish oil has a higher EPA concentration. Thus in one embodiment the present composition further comprises at least one lipid selected from the group consisting of fish oil, marine oil, algal oil, fungal oil and microbial oil.

Lipid Globule Size

According to the present invention, lipid is present in the nutritional composition in the form of lipid globules, emulsified in the aqueous phase.

In one embodiment, preferably the lipid globules are large in size. Preferably in one embodiment according to the present invention, the lipid globules have
1) a volume-weighted mode diameter above 1.0 μm preferably above 3.0 μm more preferably 4.0 μm or above, preferably between 1.0 and 10 μm more preferably between 2.0 and 8.0 μm even more preferably between 3.0 and 8.0 μm most preferably between 4.0 μm and 8.0 μm and/or
2) a size distribution in such a way that at least 45 volume %, preferably at least 55 volume %, even more preferably at least 65 volume %, even more preferably at least 75 volume % has a diameter between 2 and 12 p.m. More preferably at least 45 volume %, preferably at least 55 volume %, even more preferably at least 65 volume %, even more preferably at least 75 volume % has a diameter between 2 and 10 p.m. Even more preferably at least 45 volume %, preferably at least 55 volume %, even more preferably at least 65 volume %, even more preferably at least 75 volume % has a diameter between 4 and 10 μm.

In another embodiment the lipid globules comprise a core and preferably a coating. The core preferably comprises vegetable fat and preferably comprises at least 90 wt. % triglycerides and more preferably essentially consists of triglycerides. The coating comprises phospholipids and/or polar lipids. Not all phospholipids and/or polar lipids that are present in the composition need necessarily be comprised in the coating, but preferably a major part is. Preferably more than 50 wt. %, more preferably more than 70 wt,%, even more preferably more than 85 wt. %, most preferably more than 95 wt. % of the phospholipids and/or polar lipids that are present in the composition are comprised in the coating of lipid globules. Not all vegetable lipids that are present in the composition need necessarily be comprised in the core of lipid globules, but preferably a major part is, preferably more than 50% wt. %, more preferably more than 70 wt. %, even more preferably more than 85 wt. %, even more preferably more than 95 wt. %, most preferably more than 98 wt. % of the vegetable lipids that are present in the composition are comprised in the core of lipid globules. In one embodiment the lipid globules of the present invention preferably have a coating comprising phospholipids, the phospholipids preferably being present in an amount of 0.5 to 20 wt. % based on total lipid of the nutritional composition and the lipid globules have
1) a volume-weighted mode diameter above 1.0 μm, preferably above 3.0 μm, more preferably 4.0 μm or above, preferably between 1.0 and 10 μm, more preferably between 2.0 and 8.0 μm, even more preferably between 3.0 and 8.0 μm, most preferably between 4.0 μm and 8.0 μm and/or
2) a size distribution in such a way that at least 45 volume %, preferably at least 55 volume %, even more preferably at least 65 volume %, even more preferably at least 75 volume % has a diameter between 2 and 12 μm. More preferably at least 45 volume %, preferably at least 55 volume %, even more preferably at least 65 volume %, even more preferably at least 75 volume % has a diameter between 2 and 10 μm. Even more preferably at least 45 volume %, preferably at least 55 volume %, even more preferably at least 65 volume %, even more preferably at least 75 volume % has a diameter between 4 and 10 μm.

In another preferred embodiment the lipid globules of the present invention preferably have a coating comprising phospholipids, the phospholipids preferably being present in an amount of 0.5 to 20 wt. % based on total lipid of the nutritional composition and the lipid globules have
1) a volume-weighted mode diameter below 1.0 μm, and preferably in the range of 0.2-0.7 μm, more preferably in the range of 0.3-0.6 μm, and
2) a size distribution in such a way that less than 45 volume %, has a diameter between 2 and 12 μm, preferably a size distribution wherein more than 55 volume % of the lipid globules has a diameter of less than 2 μm, more preferably a size distribution in such a way that less than 35 volume %, has a diameter between 2 and 12 μm, even more preferably a size distribution wherein more than 65 volume % of the lipid globules has a diameter of less than 2 μm.

The percentage of lipid globules is based on volume of total lipid. The mode diameter relates to the diameter which is the most present based on volume of total lipid, or the peak value in a graphic representation, having on the X-as the diameter and on the Y-as the volume (%).

The volume of the lipid globule and its size distribution can suitably be determined using a particle size analyzer such as a Mastersizer (Malvern Instruments, Malvern, UK), for example by the method described in Michalski et al, 2001, Lait 81: 787-796.

Process for Obtaining Polar Lipid Coated Lipid Globules

The present composition comprises lipid globules. The lipid globule size can be manipulated by adjusting process steps by which the present composition is manufactured. A suitable and preferred way to obtain lipid globules coated with polar lipids is to increase the amount of polar lipids compared to amounts typically present in infant formula and to have these polar lipids present during the homogenization process, wherein the mixture of aqueous phase and oil phase is homogenized. WO 2010/027258 and WO 2010/027259 describe examples of such processes. A typical amount of phospholipids is about 0.15 wt. % based on total fat and a typical amount of polar lipids in infant formula is about 0.2 wt. % based on total fat. For a better coating of the lipid globules the amount of phospholipids is increased to at least 0.5 wt %, more preferably at least 1.0 wt. % based on total fat or the amount of polar lipids is increased to at least 0.6 wt. %, more preferably at least 1.0 wt. % based on total fat.

Digestible Carbohydrate Component

The composition preferably comprises digestible carbohydrate. The digestible carbohydrate preferably provides 30 to 80% of the total calories of the composition. Preferably the digestible carbohydrate provides 40 to 60% of the total calories. When in liquid form, e.g. as a ready-to-feed liquid, the composition preferably comprises 3.0 to 30 g digestible carbohydrate per 100 ml, more preferably 6.0 to 20, even more preferably 7.0 to 10.0 g per 100 ml. Based on dry weight the present composition preferably comprises 20 to 80 wt. %, more preferably 40 to 65 wt. % digestible carbohydrates.

Preferred digestible carbohydrate sources are lactose, glucose, sucrose, fructose, galactose, maltose, starch and maltodextrin. Lactose is the main digestible carbohydrate present in human milk. The present composition preferably comprises lactose. The present composition preferably comprises digestible carbohydrate, wherein at least 35 wt. %, more preferably at least 50 wt. %, more preferably at least 75 wt. %, even more preferably at least 90 wt. %, most preferably at least 95 wt. % of the digestible carbohydrate is lactose. Based on dry weight the present composition preferably comprises at least 25 wt. % lactose, preferably at least 40 wt. %.

Non-Digestible Oligosaccharides

Preferably the present composition comprises non-digestible oligosaccharides with a degree of polymerization (DP) between 2 and 250, more preferably 3 and 60. The non-digestible oligosaccharides advantageously improve intestinal microbiota.

The non-digestible oligosaccharide is preferably selected from the group consisting of fructo-oligosaccharides (such as inulin), galacto-oligosaccharides (such as transgalacto-oligosaccharides or beta-galacto-oligisaccharides), gluco-oligosaccharides (such as gentio-, nigero- and cyclodextrin-oligosaccharides), arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, sialic acid comprising oligosaccharides and uronic acid oligosaccharides. Preferably the composition comprises gum acacia in combination with a non-digestible oligosaccharide.

Preferably the present composition comprises fructo-oligosaccharides, galacto-oligosaccharides and/or galacturonic acid oligosaccharides, more preferably galacto-oligosaccharides, most preferably transgalacto-oligosaccharides. In a preferred embodiment the composition comprises a mixture of transgalacto-oligosaccharides and fructo-oligosaccharides. Preferably the present composition comprises galacto-oligosaccharides with a DP of 2-10 and/or fructo-oligosaccharides with a DP of 2-60. The galacto-oligosaccharide is preferably selected from the group consisting of transgalacto-oligosaccharides, lacto-N-tetraose (LNT), lacto-N-neotetraose (neo-LNT), fucosyl-lactose, fucosylated LNT and fucosylated neo-LNT. In a particularly preferred embodiment the present invention comprises the administration of transgalacto-oligosaccharides ([galactose]ₙ-glucose; wherein n is an integer between 1 and 60, i.e. 2, 3, 4, 5, 6, . . . , 59, 60; preferably n is selected from 2, 3, 4, 5, 6, 7, 8, 9, or 10). Transgalacto-oligosaccharides (TOS) are for example sold under the trademark Vivinal™ (Borculo Domo Ingredients, Netherlands). Preferably the saccharides of the transgalacto-oligosaccharides are β-linked.

Fructo-oligosaccharide is a non-digestible oligosaccharide comprising a chain of β linked fructose units with a DP or average DP of 2 to 250, more preferably 10 to 100. Fructo-oligosaccharide includes inulin, levan and/or a mixed type of polyfructan. An especially preferred fructo-oligosaccharide is inulin. Fructo-oligosaccharide suitable for use in the compositions is also already commercially available, e.g. Raftiline®HP (Orafti).

Uronic acid oligosaccharides are preferably obtained from pectin degradation. Uronic acid oligosaccharides are preferably galacturonic acid oligosaccharides. Hence the present composition preferably comprises a pectin degradation product with a DP between 2 and 100. Preferably the pectin degradation product is prepared from apple pectin, beet pectin and/or citrus pectin. Preferably the composition comprises transgalacto-oligosaccharide, fructo-oligosaccharide and a pectin degradation product. The weight ratio transgalacto-oligosaccharide:fructo-oligosaccharide:pectin degradation product is preferably (20 to 2):1:(1 to 3), more preferably (12 to 7):1:(1 to 2).

Preferably, the composition comprises of 80 mg to 2 g non-digestible oligosaccharides per 100 ml, more preferably 150 mg to 1.50 g, even more preferably 300 mg to 1 g per 100 ml. Based on dry weight, the composition preferably comprises 0.25 wt. % to 20 wt. %, more preferably 0.5 wt. % to 10 wt. %, even more preferably 1.5 wt. % to 7.5 wt. %. A lower amount of non-digestible oligosaccharides will be less effective in providing a beneficial prebiotic effect, whereas a too high amount will result in side-effects of bloating and abdominal discomfort.

Protein Component

The present composition preferably comprises proteins. The protein component preferably provides 5 to 15% of the total calories. Preferably the present composition comprises a protein component that provides 6 to 12% of the total calories. More preferably protein is present in the composition below 9% based on total calories, more preferably the composition comprises between 7.2 and 8.0% protein based on total calories, even more preferably between 7.3 and 7.7% based on total calories. The protein concentration in a nutritional composition is determined by the sum of protein, peptides and free amino acids. Based on dry weight the composition preferably comprises less than 12 wt. % protein, more preferably between 9.6 to 12 wt. %, even more preferably 10 to 11 wt. %. Based on a ready-to-drink liquid product the composition preferably comprises less than 1.5 g protein per 100 ml, more preferably between 1.2 and 1.5 g, even more preferably between 1.25 and 1.35 g.

The source of the protein preferably is selected in such a way that the minimum requirements for essential amino acid content are met and satisfactory growth is ensured. Hence protein sources based on cows' milk proteins such as whey, casein and mixtures thereof and proteins based on soy, potato or pea are preferred. In case whey proteins are used, the protein source is preferably based on acid whey or sweet whey, whey protein isolate or mixtures thereof and may include α-lactalbumin and β-lactoglobulin. More preferably, the protein source is based on acid whey or sweet whey from which caseino-glyco-macropeptide (CGMP) has been removed. Removal of CGMP from sweet whey protein advantageously reduces the threonine content of the sweet whey protein. A reduced threonine content is also advantageously achieved by using acid whey. Optionally the protein source may be supplemented with free amino acids, such as methionine, histidine, tyrosine, arginine and/or tryptophan in order to improve the amino acid profile. Preferably α-lactalbumin enriched whey protein is used in order to optimize the amino acid profile. Using protein sources with an optimized amino acid profile closer to that of human breast milk enables all essential amino acids to be provided at reduced protein concentration, below 9% based on calories, preferably between 7.2 and 8.0% based on calories and still ensure a satisfactory growth. If sweet whey from which CGMP has been removed is used as the protein source, it is preferably supplemented by free arginine in an amount of from 0.1 to 3 wt. % and/or free histidine in an amount of from 0.1 to 1.5 wt. % based on total protein.

Preferably the composition comprises at least 3 wt. % casein based on dry weight. Preferably the casein is intact and/or non-hydrolyzed.

Nutritional Composition

The present composition is preferably particularly suitable for providing the daily nutritional requirements to a human with an age below 36 months, particularly an infant with the age below 24 months, even more preferably an infant with the age below 18 months, most preferably below 12 months of age. Hence, the nutritional composition is for feeding or is used for feeding a human subject. The present composition comprises a lipid, and preferably a protein and preferably a digestible carbohydrate component wherein the lipid component preferably provides 30 to 60% of total calories, the protein component preferably provides 5 to 20%, more preferably 5 to 15 wt. %, of the total calories and the digestible carbohydrate component preferably provides 25 to 75% of the total calories. Preferably the present composition comprises a lipid component providing 35 to 55% of the total calories, a protein component provides 6 to 12% of the total calories and a digestible carbohydrate component provides 40 to 60% of the total calories. The amount of total calories is determined by the sum of calories derived from protein, lipids and digestible carbohydrates. Protein and carbohydrates are considered to provide a caloric density of 4 kcal/g and lipid of 9 kcal/g.

The present composition is not human breast milk. The present composition comprises vegetable lipids. The compositions of the invention preferably comprise other fractions, such as vitamins, minerals according to international directives for infant formulae.

In one embodiment the composition is a powder suitable for making a liquid composition after reconstitution with an aqueous solution, preferably with water. Preferably the composition is a powder to be reconstituted with water. It was surprisingly found that the size and the coating with polar lipids of the lipid globules remained the same after the drying step and subsequent reconstitution.

In order to meet the caloric requirements of the infant, the composition preferably comprises 50 to 200 kcal/100 ml liquid, more preferably 60 to 90 kcal/100 ml liquid, even more preferably 60 to 75 kcal/100 ml liquid. This caloric density ensures an optimal ratio between water and calorie consumption. The osmolarity of the present composition is preferably between 150 and 420 mOsmol/l, more preferably 260 to 320 mOsmol/l. The low osmolarity aims to reduce the gastrointestinal stress.

Preferably the composition is in a liquid form, with a viscosity below 35 mPa·s, more preferably below 6 mPa·s as measured in a Brookfield viscometer at 20° C. at a shear rate of 100 s⁻¹.

Suitably, the composition is in a powdered from, which can be reconstituted with water to form a liquid, or in a liquid concentrate form, which should be diluted with water. When the composition is in a liquid form, the preferred volume administered on a daily basis is in the range of about 80 to 2500 ml, more preferably about 450 to 1000 ml per day.

Infant

The composition of the present invention is preferably for use in infants. Because of the benefits for the developing child, it is advantageous to establish the present energy expenditure programming effect early in life. Hence the present composition is preferably administered to the human subject during the first 3 years of life. In one embodiment according to the present invention the nutritional composition is provided to the human subject when the human subject has an age of 0 to 12 months. In one embodiment according to the present invention, the nutritional composition is for feeding or is used for feeding a human subject with an age between 0 and 36 months. The present composition is advantageously administered to a human of 0 to 24 months, more preferably to a human of 0 to18 months, most preferably to a human of 0 to 12 months.

In one embodiment according to the present invention the nutritional composition is adminstered to a human subject that has an age of 0 to 36 months and that is at risk of developing metabolic disease later in life and/or developing diabetes type 2 later in life.

Preferably the composition is to be used in infants which are prematurely born or which are small for gestational age. These infants experience after birth a catch up growth, which requires extra attention on body composition development. Thus in one embodiment the nutritional composition is adminstered to a human subject that has an age of 0 to 36 months and that is at risk of developing metabolic disease later in life and/or developing diabetes type 2 later in life and the human subject is selected from the group consisting of infants born with a birth weight below 1500 gram and/or infants born before week 37 of gestation. Preferably the composition is to be used in infants which are large for gestational age, since in these infants are at risk for higher weight gain during the first year of life. Thus in one embodiment the nutritional composition is adminstered to a human subject that has an age of 0 to 36 months and that is at risk of developing metabolic disease later in life and/or developing diabetes type 2 later in life and the human subject is born with a birth weight above 4200 gram.

Moreover there is evidence that first degree relatives of diabetes type 2 patients have altered mitochondrial number and function. Offspring of mothers with diabetes type 2 have a decreased numer of mitochondrial number and activity. Therefore preferably the composition is to be used in infants born from mothers with diabetes type 2 or with gestational diabetes. Thus in one embodiment the nutritional composition is adminstered to a human subject that has an age of 0 to 36 months and that is born from a mother with diabetes type 2 or from a mother with gestational diabetes. In one embodiment the human subject has an age of 0 to 36 months and that is born from a mother with diabetes type 2 or from a mother with gestational diabetes, is at risk of developing diabetes type 2 later in life.

Application

The inventors surprisingly found that feeding during infance a diet with a fat component with large lipid globules coated with phospholipids, resulted in a higher energy expenditure during exposure to a high fat, high energy Western style diet later on.

Total energy expenditure (TEE) is the amount of energy (in calories or kJ) that a subject utilizes. In adults, it is the sum of the energy needed for cellular processes, physical activity (exercise and other physical activity) and internal heat produced (i.e. thermogenesis). In children additional energy is needed for growth.

For the present purpose, the baseline measures of energy expenditure—basal and resting energy expenditure (BEE and REE, respectively) are the most relevant. When measured on quiescent individuals, at a common temperature and corrected for body mass, these estimate the compulsory energy cost of self-maintenance. BEE, also referred to a basal metabolic rate (BMR) is the lowest rate of metabolism, measured at a particular temperature, in an inactive and post-absorptive state in specifically endothermic organisms (including mammals and birds, e.g. organisms capable of thermoregulation). REE also referred to as resting metabolic rate (RMR) also assumes a post-absorptive state, but is frequently applied to both endotherms and ectotherms and caters for low levels of spontaneous activity. Since all three measures represent the minimal metabolism of an individual in a relatively quiescent state, we group them under the term RMR or REE. Typically in humans RMR accounts to 70% of the TEE.

Thermogenesis is the production of heat by the body. It can be caused through exercise-associated thermogenesis (EAT) and non-exercise associated thermogenesis (NEAT)

When consuming a diet of the present invention early in life a higher energy expenditure, in particul RMR, in particular thermogenesis, more in particular NEAT was observed later in life.

The mechanism to increase heat in the body is by futile cycles. The most contributing futile cycle occurs in mitochondria by uncoupling oxidative phosphorylation, in other words by dissipating the energy of the proton motive force generated across the inner mitochondrial membrane as heat rather than in ATP production. Uncoupling proteins (UCPs) are involved in this. An increased activity of UCP will result in increased thermogenesis. UCP1, thermogenin, is found in brown adipose tissue (BAT), and is relevant in infants. However, recently also BAT adipocytes also are thought to play a role in adult humans. To a lower extent UCP1 is also expressed in skeletal muscle. UCP3, mainly found in skeletal muscle and white adipose tissue (WAT) is suggested to play a role in fatty acid metabolism. UCP1 and/or UCP3 overexpression protects against dietary fat induced obesity, insulin resistance and metabolic syndrome. Uncoupling oxidative phosphorylation from ATP synthesis results in increased fat oxidation, by beta-oxidation (rather than the fat being synthesized and stored in adipose tissue as energy reserve).

Mitochondria are responsible for the production of energy in the form of ATP by oxidative phosphorylation, a process in which nutrients are oxidized to form ATP. A higher mitochondrial activity protect against ectopic fat accumulation and insulin resistance. When consuming a diet of the present invention early in life a higher mitochondrial activity in both white adipose tissue (WAT) and skeletal muscle was observed later in life. This is indicated by a higher citrate synthase (CS) activity in the retroperitoneal (RP) WAT and the skeletal muscle, a higher UCP3 expression in both the RP WAT and skeletal muscle as well as a higher PDK4 expression in the RP WAT. CS activity as well as increased OXPHOS protein expression and mtDNA content is a marker for both mitochondrial content and functionality. CS activity is especially a marker for the mitochondrial density, i.e. the number of mitochondria per cell (Larsen et al; Biomarkers of mitochondrial content in skeletal muscle of healthy young human subjects; J Physiol. 2012, 590(Pt 14): 3349-60.)

Uncoupling protein 3 (UCP3) has been suggested to play a role in fatty acid metabolism and to protect against lipid induced mitochondrial dysfunction or oxidative stress and to be indirectly involved in adaptive thermogenesis. In fasted state PDK4 inhibits the pyruvate dehydrogenase complex, which oxidizes pyruvate to Acetyl CoA, and in this way inhibits the glycolysis. Increased PDK4 activity after 4 h of fasting is indicative for a faster switch from glucose to fatty acid oxidation, and hence indicative for metabolic flexibility. The increased protein expression of 5 subunits of the oxidative phosphorilation complex (OXPHOS) as a consequence of aWestern Style diet challenge further sustains this finding. The increased OXPHOS protein expression is indicative for a higher mitochondrial capacity to handle the fat challenge: fat is rather burned than stored in the WAT.

Weight control in adult, i.e. full grown, subjects is the result of an imbalance between energy intake and energy expenditure. If energy intake exceeds energy expenditure the result will be weight gain, in the form of adipose tissue mass, eventually resulting into atopic fat storage and lipotoxicity. Prevention of obesity is therefore not solely determined by affecting energy expenditure, but is also related by a too high energy intake relative to needs. In contrast, increased energy expenditure due to a higher mitochondrial activity and/or increased thermogenesis results in less atopic fat and protects against obesity. Additionally, it will have other beneficial effects on health than preventing obesity, for example improving insulin sensitivity.

Thus in summary, upon ingestion early in life of the nutrtional composition as defined herein, later in life i) energy expenditure is increased, preferably resting energy expenditure is increased, thermogenesis is increased and/or non-exercise associated thermogenesis is increased ii) expression and/or activity of (a) uncoupling protein, preferably UCP1 and/or UCP3, is increased, (b) pyruvate dehydrogenase kinase-isozyme 4 (PDK4) is increased, (c) citrate synthase (CS) is increased and/or (d) oxidative phosphorylation complex (OXPHOS) is increased, iii) mitochondrial density is increased and/or metabolic flexibility is increased.

In one embodiment of the present invention the method or use is for preventing metabolic syndrome and/or diabetes type 2 later in life.

The present composition is preferably administered orally. The present invention is preferably considered to be of benefit for human subjects at the age above 36 months. In one embodiment the present invention is for achieving the effects described herein when said human subject has an age above 36 months, preferably when said human subject has an age above 5 years, particularly above 13 years, more particularly above 18 years. In one embodiment the present invention is for feeding a human subject with an age between 0 and 36 months and for achieving the effects described herein when said human subject has an age above 36 months, preferably when said human subject has an age above 5 years, particularly above 13 years, more particularly above 18 years. In one embodiment the present invention is for i) increasing energy expenditure and preferably energy expenditure is selected form the group consisting of increasing resting energy expenditure. increasing thermogenesis and increasing non-exercise associated thermogenesis, ii) increasing expression and/or activity of (a) uncoupling protein, preferably of UCP1 and/or UCP3, (b) pyruvate dehydrogenase kinase-isozyme 4 (PDK4) and/or (c) citrate synthase (CS), iii) increasing metabolic flexibility in a human subject, when said human subject has an age above 36 months, preferably when said human subject has an age above 5 years, particularly above 13 years, more particularly above 18 years. In one embodiment the present invention is for feeding a human subject with an age between 0 and 36 months and for i) increasing energy expenditure and preferably energy expenditure is selected from the group consisting of increasing resting energy expenditure. increasing thermogenesis and increasing non-exercise associated thermogenesis, ii) increasing expression and/or activity of (a) uncoupling protein, preferably of UCP1 and/or UCP3, (b) pyruvate dehydrogenase kinase-isozyme 4 (PDK4) and/or (c) citrate synthase (CS), iii) increasing metabolic flexibility in a human subject, when said human subject has an age above 36 months, preferably at the age above 5 years, particularly above 13 years, more particularly above 18 years. In one embodiment i) increasing energy expenditure, preferably selected form the group consisting of increasing resting energy expenditure. increasing thermogenesis and increasing non-exercise associated thermogenesis, ii) increasing expression and/or activity of (a) uncoupling protein, preferably of UCP1 and/or UCP3, (b) pyruvate dehydrogenase kinase-isozyme 4 (PDK4) and/or (c) citrate synthase (CS), iii) increasing metabolic flexibility occurs later in life. With later in life is meant an age exceeding the age at which the diet is taken, preferably with at least one year. Thus in one embodiment according to the invention, the time period between providing the nutritional composition and the increase in energy expenditure, increase in expression and/or activity of (1) UCP, (2) PDK4 and/or (3) CS, or increase in metabolic flexibility is at least 12 months.

As described above, the present invention is for achieving the effects described herein when said human subject has an age above 36 months. The effects of i) increasing energy expenditure and preferably energy expenditure is selected form the group consisting of increasing resting energy expenditure. increasing thermogenesis and increasing non-exercise associated thermogenesis, ii) increasing expression and/or activity of (a) uncoupling protein, preferably of UCP1 and/or UCP3, (b) pyruvate dehydrogenase kinase-isozyme 4 (PDK4) and/or (c) citrate synthase (CS), iii) increasing metabolic flexibility are preferably achieved in human subjects that are exposed to ‘Western’ food. The increased protein expression of 5 subunits of the oxidative phosphorylation complex (OXPHOS) as a consequence of the Western Style diet challenge further supports this. The increased OXPHOS protein expression is indicative for a higher mitochondrial capacity to handle the fat challenge: fat is rather burned than stored in the WAT.

For sake of clarity it is noted that this does not necessarily mean only human subjects in the

Western world, but also to the increasing amount of subjects that are outside the Western world with ample supply to Western food. Or to put it differently, human subjects who are undernourished or live in circumstances where they have no access to Western food and human subjects that are on a strict diet and human subjects that are on healthy, e.g. low fat, diets or have healthy diet habits are excluded from human subjects that are exposed to ‘Western’ food.

It is well known by the person skilled in the art what a Western type diet is. It is high in calories, high in fat and high in sugar. The fat is high in saturated fat, it has a high n-6/n3 fatty acid ratio and is high in cholestrol. The diet is generally characterized by a high intake in processed meat, red meat, butter, high fat dairy products, sugar and refined grains. WHO/FAO has guidelines for the recommended diet and the Western diet is deviating from that guidelines, FAO (Food and Agriculture Organization of the United Nations) Food and Nutrition Paper 91: Fats and fatty acids in human nutrition—Report of an expert consultation, held 10-14 Nov. 2008 in Geneva, available in print November 2010, ISBN 978-92-5-106733-8. Western type diet is sometimes also referred to as Standard American Diet. For the purpose of the present invention, Western food or in other words a Western style diet is preferably characterised by 1) that over 30% of the total calories is provided by fat, 2) that it comprises at least 10 wt. % saturated fat based on total amount of fat, 3) that it comprises at least 0.5 wt % cholesterol based on total fat, 4) that the n6/n3 ratio of the fatty acids in the dietary fat is above 4, and in an improved definition the n6/n3 ratio of the fatty acids in the dietary fat is above 10.

It should be noted that the present nutrition for human subjects with an age of 36 months or lower differs from a the diet recommended for adults and children above 5 years of age, in several respects, because of the different need for a growing and developing body. For example, the caloric contribution (energy %) of fat in infant nutrition should be high, whereas it should be low in an adult diet.

Thus according to one embodiment of the present invention the human subject that has an age above 36 month is exposed to a high fat Western style diet.

The increase in energy expenditure is not observed at the moment the nutritional composition is provided, hence there is no direct diet effect. Thus in one embodiment according to the invention, the increase in energy expenditure does not take place when providing the nutritional composition to the human subject when the subject has an age of 0 to 36 months, more preferably 0 to 12 months.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one.

### EXAMPLES

### Example 1

### Effect of Fat Component in Early in Life Diet on Energy Expenditure Later in Life

Adult (4 months) guinea pigs (GP), obtained from Harlan Laboratories B.V. (Horst, the Netherlands), 6 males and 12 females, were acclimatized for two weeks and then mated 1:2. During the mating period GP were fed a breeding diet, containing 23% protein and 7% fat. After 6 weeks all dams were visibly pregnant and the males were separated from the females. Before birth, pregnant dams where housed individually.

Piglets were assigned to either diet 1 (control) (n=11) or diet 2 (experimental diet) (n=11). Diet 1: Control diet comprising infant formula delivering the fat. Fat component was in form of small lipid globules. The diet was the same as experimental diet 1 described in example 3 of WO 2010/027258. In short the volumetric mode diameter was 0.5 p.m and the amount of phospholipids (soy lecithin) was about 0.2 wt % based on total fat.

Diet 2: Experimental diet comprising infant formula delivering the fat. Fat component was in form of large lipid globules coated with milk derived phospholipids. The diet was the same as experimental diet 4 described in example 3 of WO 2010/027258. In short the volumetric mode diameter was 4.3 μm and the amount of phospholipids, mainly milk derived, was about 1.25 wt % based on total fat.

The diet was provided as dough and was made granular so the piglets could eat it properly. The piglets had access to the diet from postnatal day 2 (PN2) onwards but were also able to suckle until weaning at PN 21. Piglets continued their respective diets until PN42. From then all the animals were changed to a Western style diet (WSD) comprising 15 wt % fat, 18 wt % protein, 57 wt % digestible carbohydrates, 5 wt % fiber, the rest being minerals, vitamins and traces of water.

The fat component consisted of 80 wt % of soy oil and of 20 wt % of lard. The amount of cholesterol was 1 wt % based on total fat, the amount of saturated fatty acids (SFA) was 39 wt % based on total fatty acids, the amount of mono-unsaturated fatty acids (MUFA) was 39 wt % based on total fatty acids and the amount of poly-unsaturated fatty acids (PUFA) was 22 wt % based on total fatty acids. The LA/ALA and n6/n3 weigth ratio were 11.

At PN21 a temperature transponder was injected subcutaneously under general anesthesia, afterwards body temperature was recorded twice a week with a PLEXX reader (Plexx B.V., Elst, The Netherlands). GP were also weighed twice a week individually and from PN21 onwards. At PN 42, and 140 body composition was measured with a DEXA scan (Hologic Inc., Discovery A) under inhalation anesthesia (air/isoflurane) after a 3-4 hours fast. At PN42, also a 1 ml blood sample was drawn by hart puncture. At PN140, all GP were euthanized and dissected.

Table 1 shows the results of the body temperature in ° C. and the body weight. Before (data not shown) and at PN42 no difference in body temperature was observed between the two diet groups. After PN42 until PN140, the end of the experiment, body temperature was increased in animals previously adminstered diet 2 early in life. At specific time points this effect was statistically significant. Also taking into account the whole period of PN42 to PN140 the body temperature of animals of diet group 2 was significantly increased compared to animals of diet group 1. No statistically significant effect on body weight and no significant effect on body composition (lean body mass (LBM), fat mass) was observed between the 2 groups at day 42 and day 140 (data not shown). PN140 for Guinea pigs relates to young adulthood in a human setting.

TABLE 1
Temperature and body weight as measured in
the Guinea pigs previously administered diet 1
or 2 and exposed to the same Western style diet.

| | Temperature (° C. ± SEM) | |
|---|---|---|
| Postnatal day | Diet 1 | Diet 2 |
| 42 | 38.78 ± 0.08 | 38.98 ± 0.12 |
| 46 | 38.67 ± 0.09 | 38.81 ± 0.10 |
| 49 | 38.93 ± 0.07 | 39.21 ± 0.11* |
| 52 | 38.86 ± 0.11 | 38.95 ± 0.14 |
| 55 | 38.81 ± 0.11 | 39.28 ± 0.10** |
| 60 | 38.80 ± 0.10 | 39.01 ± 0.07 |
| 62 | 39.01 ± 0.09 | 39.16 ± 0.13 |
| 66 | 38.88 ± 0.12 | 39.24 ± 0.14 |
| 69 | 38.79 ± 0.12 | 39.14 ± 0.14 |
| 73 | 38.63 ± 0.07 | 39.06 ± 0.10** |
| 76 | 38.59 ± 0.14 | 39.15 ± 0.11** |
| 81 | 38.76 ± 0.13 | 39.00 ± 0.10 |
| 87 | 38.81 ± 0.13 | 39.00 ± 0.09 |
| 90 | 39.94 ± 0.12 | 39.07 ± 0.11 |
| 94 | 38.57 ± 0.11 | 39.21 ± 0.09* |
| 97 | 38.57 ± 0.15 | 39.21 ± 0.09** |
| 101 | 38.46 ± 0.15 | 39.08 ± 0.10** |
| 104 | 38.68 ± 0.16 | 38.90 ± 0.14 |
| 108 | 38.86 ± 0.15 | 39.07 ± 0.07 |
| 111 | 38.47 ± 0.15 | 39.20 ± 0.09*** |
| 115 | 38.56 ± 0.12 | 39.19 ± 0.15** |
| 122 | 38.60 ± 0.15 | 39.08 ± 0.12* |
| 125 | 38.49 ± 0.15 | 38.78 ± 0.11 |
| 130 | 38.67 ± 0.10 | 38.93 ± 0.15 |
| 132 | 38.69 ± 0.16 | 38.91 ± 0.09 |
| 140 | 38.59 ± 0.29 | 39.02 ± 0.14 |

*indicating significant difference p < 0.05;
**indicating significant difference p < 0.01;
***indicating significant difference p < 0.001.

The diet group 2 animals in the guinea pig study developed a higher body temperature in response to the WSD challenge compared to the diet group 1 animals, suggesting a higher thermogenesis due to higher UCP activity and/or number of mitochondria of these animals, resulting in increased energy expenditure.

### Example 2

### Effect of Fat Component in Early in Life Diet on Uncouping Protein and Mitochondrial Function Later in Life

Weaning male C57/BL6 mice pups were fed either a control diet 1 or experimental diet 2 as in example 1. Mice were exposed to the diets from PN15 onwards and fully weaned ad PN21. From PN42 until PN98 the mice were fed either AIN 93 M or challenged with a Western Style diet

(WSD), high in fat and energy. The composition was 18 wt % protein, 20 wt. % fat, 52 wt % digestible carbohydrates, 5 wt % fibers, the remaining 5% being vitamins, minerals and traces of water. The same fat source of example 1 was used. As a control group mice were exposed to control diet 1 early in life and subsequently fed standard AIN-93 based chow up to day 98 (group 3), which is not a high fat, high energy, in other words, not an obesogenic diet. Before dissection, the animals were fasted for four hours. At dissection RP WAT depots and musculus tibialis were snap frozen and stored at −80° C. until they were used for gene expression and enzyme activity analysis. NB: Energy expenditure cq thermogenesis can not be measured directly in this model in a similar way as in example 1 due to the higher physical activity of mice compared to guinea pigs.

The citrate synthase activity is analyzed as described previously (Gosker, H. et al, 2005, Am J Physiol Endocrinol Metab; 290: E976-E981). The RP WAT depots and musculis tibialis were used for RNA isolation to perform gene expression analysis. RNA was isolated by using Trizol (Invitrogen, Breda, The Netherlands) according to manufacturer's instructions, after which the RNA was purified with the RNeasy Mini Kit (Qiagen Benelux b.v., Zwijndrecht, The Netherlands) including a DNase treatment with a RNase-free DNase Set (Qiagen Benelux b.v., Zwijndrecht, The Netherlands). Quantity and quality of the RNA samples were analyzed with the Nanodrop 2000 (Thermo Scientific, Breda, The Netherlands) and the Bioanalyzer (Agilent, Santa Clara, USA) respectively. cDNA was synthesized with the iScript cDNA synthese kit (Bio-Rad, Veenendaal, The Netherlands) according to manufacturer's instructions. For the Q-PCR reaction 5× Hot FirePol Evagreen qPCR mix Plus (Bio-Connect, Huissen, The Netherlands) was used according to manufacturer's instructions and the Q-PCR was run on the 7900HT Fast Real Time PCR System (Applied Biosystems, Bleiswijk, The Netherlands). The following program was used, 2 minutes 50° C.; 10 minutes 95° C.; 40 cycli of 15 seconds 95° C. followed by 1 minute 60° C.; after which a dissociation program was performed. The primer sequences were: UCP3: aacgctcccctaggcaggta, gcagaaaggagggcacaaatc and PDK4: aagagctggtatatccagagcctg, ttgaccagcgtgtctacaaactc. UCP1: CAAAAACAGAAGGATTGCCGAAA and TCTTGGACTGAGTCGTAGAGG. RP119 and RPS29 were used as reference genes. The data was analyzed with qbase PLUS (Biogazelle, Gent, Belgium).

Data are shown in Table 2. The expresion of PDK4 and UCP3 in RP WAT and m tibialis (muscle tissue) is increased in group 2 when compared to group 1 and is more close to the values found in animals not exposed to the high fat WSD, but to normal rodent chow (diet 3).

TABLE 2
Relative mRNA expression in arbitrary units in the mouse model. The mRNA
expression is displayed as the mean expression level, scaled to the average
expression, plus the 95% CI.

| | Diet 1 | Diet 2 | Control, group 3 |
|---|---|---|---|
| PDK4 | 0.746 (0.582-0.957)* | 1.028 (0.662-1.597) | 1.402 (0.907-2.167) |
| UCP3 RP WAT | 0.862 (0.716-1.037)* | 1.006 (0.758-1.336) | 1.239 (1.077-1.424) |
| UCP3 m.tibialis | 0.812 (0.684-0.963)^{$} | 1.035 (0.893-1.198) | 1.237 (1.021-1.499) |

*significant different from group 3,
^{$}significant different from diet group 2 and 3.

There was a difference in PDK4 expression in the WAT (F_{(2,31)}=3.269; p=0.05). Diet group 1 and group 3 were significantly different from each other (p=0.02), but diet group 2 was not significantly different from both other diet groups (diet group 1: p=0.18 and group 3: p=0.22). There was a difference in UCP3 expression in the WAT (F_{(2,31)}=3.216; p=0.05). Diet group 1 and group 3 were significantly different from each other (p=0.02) but diet group 2 was not significantly different from both other diet groups (diet group 1: p=0.28 and group 3: p=0.16). There was a significant effect on the UCP3 expression in the skeletal muscle (F_{(2,30)}=7.715; p=0.002). Diet group 1 was significantly different from both diet group 2 and group 3 (p=0.03 and 0.001 respectively). Diet group 2 and the no-WSD-control were not significantly different from each other (p=0.12).

Also UCP1 expression was measured and the measured values were around detection limits. Strikingly in the group of animals having been exposed to the experimental diet 2 in 10 out of 12 animals UCP1 expression was detectable and in the animals having been exposed to the control diet 1 in 5 out of 12 animals UCP1 expression was detectable. This is indicative of an increased UCP1 expression after having consumed the diet of the present invention early in life.

Also the citrate synthase activity is increased in group 2 compared to group 1 and more close to the control raised on rodent chow and not exposed to high fat Western style diet later in life, group 3, see Table 3.

TABLE 3
Citrate synthase activity in RP WAT (U/g protein)
as measured in the mouse model (±SEM)

| | Citrate synthase activity (U/g protein) |
|---|---|
| Diet 1 | 138.326 ± 13.840 |
| Diet 2 | 213.101 ± 27.774 |
| Group 3 | 298.696 ± 34.594 |

Overall significant difference in citrate synthase activity: F_{(2,27)}=8.945; p=0.001 Diet group 1 is significantly higher than both diet group 2 and 3 (p=0.059 and 0.000 respectively).

Diet group 2 is also significant different from diet group 3 (p=0.032).

In example 1 the diet group 2 animals developed a higher body temperature in response to the WSD challenge compared to the diet group 1 animals, suggesting a higher uncoupling activity in the mitochondria of these animals. These results are now further supported by a higher UCP3 expression in both the skeletal muscle and the WAT of the diet 2 animals in the present mouse model together with a higher PDK4 activity in the WAT of the diet 2 animals in the present mouse model. The higher citrate synthase activity in the WAT of the diet 2 animals in the mouse model, implicate also a higher mitochondrial number as a consequence of early postnatal diet 2 of the present invention. Together, these data showed that early postnatal diet of the present invention results in a higher mitochondrial activity and higher mitochondrial number after a WSD challenge compared to an early in life standard infant nutrition, and in a faster switch to fat oxidation upon fasting. Interestingly and advantageously, the values obtained for UCP3 and PDK4 expression and CS activity after an early in life diet of the present invention and after being exposed to a high fat high energy diet was more like the values obtained in animals exposed to a standard, healthy non obesogenic diet.

In an experiment with similar set up and with the same diets, but with Wistar rats, CS activity was measured in the skeletal muscle m. tibialis, after having been exposed to the Western style diet. Instead of at PN 98, analysis was performed at PN 133 with N=8 per group. In animals having received diet 1 during infancy, CS activity was 121.7±23.8 (U/g protein±SEM). In animals having received diet 2, the diet of the present invention, CS activity was 203.4±26.6 (U/g protein±SEM). This difference was significant: F_{(1,12)}=5.190; p=0.04. So, not only in RP WAT, but also in skeletal muscle CS activity is increased, i.e. a higher mitochondrial activity.

In an experiment with similar set up and with the same diets, but with Wistar rats, relative UCP1 mRNA expression was measured in the brown adipose tissue (BAT), after having been exposed to the Western style diet. Instead of at PN 98, analysis was performed at PN 120 with N=8 per group. The method was as described in example 2 (Q-PCR) with the following primers: caatgaccatgtacaccaagg, agcacacaaacatgatgacg and B2M, Hprt and Rp119 as reference genes. In animals having received diet 1 during infancy, relative UCP1 mRNA expression was 0.923 (0.769-1.108) (arbitrary units, plus the 95% CI). In animals having received diet 2 during infancy, relative UCP1 mRNA expression was 1.084 (0.784-1.498) (arbitrary units, plus the 95% CI). This example confirmed the data of example 1 which shows an increased body temperature in animals fed the experimental diet.

### Example 3

### Effect of Fat Component in Early in Life Diet on Mitochondrial Content and Function Later in Life

Weaning male C57/BL6 mice pups were fed either a control diet 1 or experimental diet 2, after which the animals were challenged with a WSD. The diets had the same composition as described in Example 2. As a control group mice were exposed to control diet 1 early in life and subsequently fed standard AIN-93 based chow up to day 98 (group 3), which is not a high fat, high energy, i.e. not an obesogenic diet. Diets and study design were further as described in Example 2. At dissection RP WAT depots were snap frozen and stored at -80° C. until they were used for mitochondrial DNA (mtDNA) expression and protein expression analysis. Nuclear and mitochondrial DNA (mtDNA) was isolated from RP WAT with the QIAamp DNA micro kit (Qiagen Benelux b.v., Zwijndrecht, The Netherlands), according to manufacturers protocol. DNA quantity was determined with a Nanodrop 2000 (Thermo Scienctific, Breda, The Netherlands). 135 ng input DNA was used for each qPCR reaction (as described in example 2). Data was analyzed with qbase PLUS (Biogazelle, Genth, Belgium) and LPL was used to normalize for nuclear DNA. The primer sequences were: ND1: ACCAATACGCCCTTTAACAAC, AATGGGTGTGGTATTGGTAGG; LPL: TCCTGATGACGCTGATTTTG and ATGTCAACATGCCCTACTGG. RP depots were homogenised in RIPA buffer (Fisher Scientific, Landsmeer, The Netherlands) with protease inhibitor cocktail (Roche diagnostics, Almere, the Netherlands). Per sample a total amount of 15 μg total protein was used for SDS-PAGE. SDS PAGE gel electrophoreses was performed with the Midi-Protean TGX Precast gel 4-15%, after which proteins were transferred to a PVDF membrane with a Trans-Blot® Turbo™ Blotting System using the Trans-Blot Turbo Midi PVDF Transfer pack (Bio-Rad, Veenendaal, The Netherlands). After blocking with 5% Protifar Plus (Nutricia, Zoetermeer, The Netherland) for 1 hour, blots were incubated overnight with Mito-Profile® Total OXPHOS rodent western Blot Antibody cocktail (Abcam, Cambridge, UK) and incubated for 1 hour with ECL anti mouse IgG (Fisher Scientific, Landsmeer, The Netherlands). OXPHOS protein expression was detected with the Chemidoc XRS, analyzed by Quantity One (Biorad, Veenendaal, The Netherlands) and adjusted for total protein levels per lane, by the means of a Coomassie staining of the blot.

Data are shown in table 4. Mitochondrial content of the RP depot measured as relative mtDNA expression was higher in group 2 when compared to group 1 and is more close to the expression found in animals not exposed to the high fat WSD, but to normal rodent chow (diet 3). This data confirmed the data as found in example 2. As a result of the high fat WSD, the protein expression of 5 subunits of the oxidative phosphorylation (OXPHOS) complex was higher in group 2 when compared to group 1.

TABLE 4
Relative OXPHOS protein expression and relative mtDNA expression. OXPHOS
protein expression measured by Western Blot and corrected for total protein expression and
expressed ad mean ± SEM. mtDNA expression expressed as mean expression corrected for mean
genomic DNA expression, scaled to the average expression, plus the 95% CI.

| | Diet 1 | Diet 2 | Control, group 3 |
|---|---|---|---|
| Complex I (NDUFB8) | 0.339 ± 0.069 | 0.523 ± 0.108 | 0.394 ± 0.132 |
| Complex II (SDHB) | 1.126 ± 0.244 | 1.347 ± 0.213^{$} | 0.718 ± 0.106 |
| Complex III | 0.969 ± 0.227 | 1.472 ± 0.191 | 1.056 ± 0.165 |
| (UQCRC2) | | | |
| Complex IV | 0.589 ± 0.098 | 0.954 ± 0.135⁺ | 0.745 ± 0.085 |
| (MTCOI) | | | |
| Complex V (ATP5A) | 0.991 ± 0.171 | 1.177 ± 0.171^{#} | 0.657 ± 0.084 |
| Total OXPHOS | 0.890 ± 0.160 | 1.127 ± 0.151^{$} | 0.683 ± 0.085 |
| mtDNA expression | 1.219 (1.026-1.448)^{##} | 1.453 (1.295-1.631)⁺ | 1.908 (1.475-2.469) |

^{#}p < 0.05 different from group 3;
^{##}p < 0.01 different from group 3;
^{$}p = 0.10-0.05 different from group 3;
⁺p = 0.10-0.05 different from diet group 1.

Mitochondrial content of the RP depot, measured as relative mtDNA expression, was affected by the diet intervention (F_{(2,15)}=5.315; p=0,018). Animals of group 1 had a decreased mtDNA content compared to group 3 (p=0.005) and tended to have a decreased mtDNA content compared to group 2 (p=0.085).While no difference in mtDNA content was found between group 2 and the non-challenged control group 3. The protein expression of complex V of the OXPHOS complex was affected by the diet intervention (F_{(2,22)}=3.744; p=0.040). Group 2 had a higher complex V expression compared to the non challenged group 3 (p=0.015) and tended to have a higher expression compared to group 1 (p=0.092). The protein expression of complex II and IV and the total OXPHOS expression tended to be affected by the diet intervention (F_{(2,22)}=3.072; p=0.67; F_{(2,22)}=2.809; p=0.082 and F_{(2,22)}=2.943; p=0.074 respectively). The expression of complex II and the total OXPHOS expression tended to be increased in group 2 compared to group 3 (p=0.026 and p=0.024). The expression of complex IV tended to be increased in group 2 compared to group 1 (p=0.027).

In example 2 the diet group 2 animals showed a higher mitochondrial content in the WAT as determined by the CS activity (Larsen et al., J Physiol. 2012, 590 (Pt 14): 3349-60). These results are now further supported by a higher relative mtDNA expression in the WAT of animals fed the diet 2 in early life in the present mouse model. The present example also showed a higher oxidative capacity for the animals fed diet 2 in early life as shown by the higher expression of the OXPHOS proteins in group 2 as a consequence of the WSD challenge. This indicates an improved capability of the animals of diet group 2 to handle the fat challenge, they rather burn the fat than store this in the WAT.

### Example 4

### Effect of Fat Component in Early in Life Diet on Uncoupling Protein Expression Later in Life

Weaning male C57/BL6 mice pups were fed either a control diet 1 (group 1) as described in example 2, an experimental diet 2 as described in example 2 (group 2) or experimental diet 3 (group 3).

Experimental diet 3 comprised infant formula delivering the fat. Fat component was in form of large lipid globules without a coating of phospholipids. The diet was the same as experimental diet 2 described in example 3 of WO 2010/027258.

As a control group mice were exposed to control diet 1 early in life and subsequently fed standard AIN-93 based chow up to day 98 (group 4), which is not a high fat, high energy, i.e.not an obesogenic diet. Diets and study design were as described in Example 2. At dissection RP WAT depots were snap frozen and stored at −80° C. until they were used for gene expression analysis. UCP3 mRNA expression was determined as described in Example 2.

The expression of UCP3 tended to be increased in animals fed a diet with large lipid droplets in early life (F_{(1,61)}=3.779; p=0.057). When analyzed per diet group (data are shown in table 5) an effect was found of the diet intervention on the UCP3 expression as well (F_{(6,70)}=4.597; p=0.01). The UCP 3 expression of group 1 was decreased, while the UCP expression of group 3 was closer to the levels of the non challenged animals of group 4. This effect was even further enhanced by a diet with lipid globules comprising a coating of phospholipids (group 2). This shows that both an increased lipid droplet size and a phospholipid coating increase the UCP3 expression in the WAT, with the lipid droplet size having the highest impact.

TABLE 5
UCP3 mRNA expression in arbitrary units as a consequence
of either lipid droplet size or added phospholipids (PL) and
per diet group. The RNA expression is displayed as the mean
expression level, scaled to the average expression, plus the 95% CI.

| Diet group | UCP3 expression |
|---|---|
| Group 1 | 0.989 (0.810-1.207)^{#} |
| Group 2 | 1.149 (0.827-1.596) |
| Group 3 | 1.039 (0.838-1.289)^{#} |
| Control, group 4 | 1.445 (1.207-1.731) |

^{#}p < 0.05 different from control group 4.

In example 2 the animals of diet group 2 had an increased UCP3 expression in response to the WSD challenge and in example 1 the animals of group 2 developed an increased body temperature in response to the WSD challenge, suggesting an increased mitochondrial a higher uncoupling activity in the mitochondria of these animals. The present example shows that large lipid droplets in the diet also increases the UCP 3 expression although the combination of large lipid droplets with PL coating had the most pronounced effects.

1. A method for increasing expression and/or activity of
(1) uncoupling protein (UCP),
(2) pyruvate dehydrogenase kinase-isozyme 4 (PDK4),
(3) citrate synthase (CS) and/or
(4) oxidative phosphorylation complex
in a human subject when the human subject has reached an age above 36 months comprising providing a nutritional composition comprising lipid to the human subject when the human subject has an age of 0 to 36 months, wherein the lipid is present in the nutritional composition in an amount of at least 10 wt % based on dry weight and is in the form of lipid globules, the lipid globules having
a) a volume weighted mode diameter above 1.0 μm and/or
b) a coating of phospholipids, the phospholipids being present in an amount of 0.5 to 20 wt % based on total lipid of the nutritional composition.

2. A method for increasing mitochondrial density and/or increasing metabolic flexibility in a human subject when the human subject has reached an age above 36 months comprising providing a nutritional composition comprising lipid to the human subject when the human subject has an age of 0 to 36 months, wherein the lipid is present in the nutritional composition in an amount of at least 10 wt % based on dry weight and is in the form of lipid globules, the lipid globules having
a) a volume weighted mode diameter above 1.0 μm and/or
b) a coating of phospholipids, the phospholipids being present in an amount of 0.5 to 20 wt % based on total lipid of the nutritional composition.

3. The method according to claim 1 for increasing energy expenditure in a human subject.

4. The method according to claim 3, wherein the increased energy expenditure is selected from the group consisting of an increased resting energy expenditure, increased thermogenesis, and increased non-exercise associated thermogenesis.

5. A method for increasing energy expenditure in a human subject when the human subject has reached an age above 36 months comprising providing a nutritional composition comprising lipid to the human subject when the human subject has an age of 0 to 36 months, wherein the lipid is present in the nutritional composition in an amount of at least 10 wt % based on dry weight and is in the form of lipid globules, the lipid globules having
a) a volume weighted mode diameter above 1.0 μm and/or
b) a coating of phospholipids, the phospholipids being present in an amount of 0.5 to 20 wt % based on total lipid of the nutritional composition.

6. The method according to claim 5, wherein the increased energy expenditure is selected from the group consisting of an increased resting energy expenditure, increased thermogenesis, and increased non-exercise associated thermogenesis.

7. The method according to claim 1, wherein the human subject that has an age above 36 month is exposed to a high fat Western style diet.

8. The method according to claim 1, wherein the nutritional composition is provided to the human subject when the human subject has an age of 0 to 12 months.

9. The method according to claim 1, wherein the increase in energy expenditure does not take place when providing the nutritional composition to the human subject when the subject has an age of 0 to 36 months, more preferably 0 to 12 months.

10. The method according to claim 1, wherein the time period between providing the nutritional composition and the increase in energy expenditure, increase in expression and/or activity of (1) UCP, (2) PDK4 and/or (3) CS, or increase in metabolic flexibility is at least 12 months.

11. The method according to claim 1, for preventing metabolic syndrome and/or diabetes type 2 later in life.

12. The method according to claim 1, wherein the nutritional composition is provided to a human subject that has an age of 0 to 36 months and that is at risk of developing metabolic disease later in life and/or developing diabetes type 2 later in life, and the human subject is selected from the group consisting of infants born with a birth weight below 1500 gram, infants born before week 37 of gestation and infants born with a birth weight above 4200 gram.

13. The method according to claim 1, wherein the nutritional composition is provided to a human subject that has an age of 0 to 36 months and that is at risk of developing diabetes type 2 later in life and that is born from a mother with diabetes type 2 or from a mother with gestational diabetes.

14. The method according to claim 1, wherein the nutritional composition comprises at least 0.1 wt % sphingomyelin based on total lipid of the nutritional composition.

15. The method according to claim 1, wherein the lipid globules have a volume weighted mode diameter above 1.0 μm.

16. The method according to claim 1, wherein the nutritional composition comprises at least 50 wt % vegetable lipid, based on total lipid.

17. The method according to claim 1, wherein the nutritional composition comprises linoleic acid (LA) and alpha linoleic acid (ALA) in a weight ratio LA to ALA between 1 and 10.

18. The method according to claim 1, wherein the nutritional composition is an infant formula or follow on formula comprising a lipid component providing 35 to 55% of the total calories, a protein component providing 6 to 12% of the total calories and a digestible carbohydrate component providing 40 to 60% of the total calories.

19. The method according to claim 17, wherein the nutritional composition comprises linoleic acid (LA) and alpha linoleic acid (ALA) in a weight ratio LA to ALA between 3 and 7.

20. The method according to claim 9, wherein the increase in energy expenditure does not take place when providing the nutritional composition to the human subject when the subject has an age of 0 to 12 months.
